(19) United States
(12) Patent Application Publication
(10) Pub. No.: US 2023/0035422 A1
(43) Pub. Date: Feb. 2, 2023

(54) METHODS FOR TREATING DISEASES BY TARGETING ONCOGENIC LIPIDS

(71) Applicant: THE TRUSTEES OF COLUMBIA UNIVERSITY IN THE CITY OF NEW YORK, New York, NY (US)

(72) Inventors: Brent R. Stockwell, New York, NY (US); Tal Hirschhorn, New York, NY (US); Fereshteh Zandkarimi, New York, NY (US); Arie Zask, New York, NY (US)

(21) Appl. No.: 17/958,741

(22) Filed: Oct. 3, 2022

Related U.S. Application Data
(63) Continuation of application No. PCT/US2021/028634, filed on Apr. 22, 2021.
(60) Provisional application No. 63/015,156, filed on Apr. 24, 2020.

(51) Int. Cl. A61K 31/4706; A61P 35/00; A61K 31/517; A61K 31/655; A61K 31/455; A61K 31/437; A61K 31/18; A61K 31/4453; A61K 31/357; A61K 33/243; A61K 31/495; A61K 31/704; A61K 31/675; A61K 31/519; A61K 31/513; A61K 31/475; A61K 31/337; A61K 38/14; A61K 31/573; A61K 39/395

(52) U.S. Cl. CPC A61K 31/4706; A61K 31/18; A61K 31/337; A61K 31/357; A61K 31/437; A61K 31/455; A61K 31/475; A61K 31/495; A61K 31/513; A61K 31/517; A61K 31/519; A61K 31/573; A61K 31/655; A61K 31/675; A61K 31/704; A61K 31/4453; A61K 33/243; A61K 38/14; A61K 39/3955; A61P 35/00

(57) ABSTRACT

The present disclosure provides, inter alia, methods for treating diseases, e.g., a cancer, in a subject by targeting oncogenic lipids in cells, including increasing lipid-based reactive oxygen species (ROS) by inhibiting coenzyme Q₁₀ (CoQ₁₀) production. Methods for treating a subject with a cancer that is sensitive to an oncolipid-targeting therapy, e.g., ADCK3 inhibition, are also provided. Further provided are methods for modulating coenzyme Q₁₀ (CoQ₁₀) level in a subject, including determining CoQ₁₀ levels by LC-MS.

### CROSS REFERENCE TO RELATED APPLICATIONS

The present application is a continuation of PCT International Application No. PCT/US2021/028634, filed Apr. 22, 2021, which claims benefit of U.S. Provisional Pat. Application Serial No. 63/015,156, filed on Apr. 24, 2020, which applications are incorporated by reference herein in their entireties.

### FIELD OF DISCLOSURE

The present disclosure provides, inter alia, methods for treating diseases, e.g., a cancer, by targeting oncogenic lipids in cells.

### GOVERNMENT FUNDING

This invention was made with government support under grant no. CA209896, awarded by the National Institutes of Health. The government has certain rights in the invention.

### BACKGROUND OF THE DISCLOSURE

In 2018 there were more than 2,000 ,000 new cases of breast cancer worldwide or 11.6% of all new cancer diagnoses (Turunen et al. 2004). As of January 2019 there are more than 3.8 million women with a history of breast cancer in the U.S., including an estimated 319,370 new cases of breast cancer in 2019 alone (Mitchell, 1975). 10-20% of breast cancers are triple-negative (Echtay et al. 2000), which translates to 31,000-63,000 or more new cases of triple-negative breast cancer in the U.S. each year, and about 200,000-400,000 new cases worldwide.

Triple-negative breast cancer is typically treated with a combination of surgery, chemotherapy, and radiation (Echtay et al. 2000). In 2019, Tecentriq (atezolizumab) was approved in combination with protein-bound paclitaxel for patients with unresectable triple-negative breast cancer whose tumors express PD-L1 (Fontaine et al. 1998). However, treatment only extended median progression-free survival by 2.6 months (from 4.8 to 7.4) and greater than 20% of patients experienced adverse reactions including alopecia, peripheral neuropathies, fatigue, nausea, diarrhea, anemia, constipation, cough, headache, neutropenia, vomiting, and decreased appetite (Fontaine et al. 1998). A number of drugs for triple-negative breast cancer (with various molecular targets) are currently in Phase I-III clinical trials. Other molecular targets with programs in preclinical or Phase I studies include iNOS, BET, COX2, TGF-beta, PIK, Aurora, TTK, NIMA, Src, Notch, Jagged, Aquaporin 1, WNT, CSF-1R, and CSPG4 (Hildebrandt and Grieshaber, 2008).

The majority of targeted cancer therapies aim to disrupt specific dysregulated signaling pathways that enhance tumor cell growth or allow tumors to overcome tumor-suppressive mechanisms inducing apoptotic cell death. Targeted therapies, such as aromatase inhibitors against estrogen receptor, and trastuzumab, which targets HER2 (ErbB2), are effective only for some breast cancer patients with subtypes that overexpress these targets. Unfortunately, no targeted therapies have been approved for triple-negative breast cancer. In addition, even with clinically successful targeted therapies, de novo and acquired resistance are major issues. Thus, identification of novel targets and induction of alternative death pathways in breast cancer tumors is of high urgency. While targeting addiction to signaling pathways has been clinically effective (e.g., BRAF, BCR-ABL, EGFR), targeting of addiction to lipid biosynthesis to induce cell death may be a new approach to cancer drug discovery.

### SUMMARY OF THE DISCLOSURE

The present disclosure provides a new therapeutic approach for targeting a specific lipid biosynthesis pathway, CoQ₁₀, to selectively sensitize a subset of breast cancers to ROS-induced ferroptosis. This can be a therapeutic solution to breast cancer patients harboring ADCK3 amplification, as a single therapy and in combination with current therapies (e.g., as a complementary treatment to apoptosis-inducing drugs, to reduce resistance and recurrence).

Accordingly, one embodiment of the present disclosure is a method for treating or ameliorating the effects of a disorder in a subject, comprising administering to the subject an effective amount of an agent that increases lipid-based reactive oxygen species (ROS).

Another embodiment of the present disclosure is a method for treating a subject with a cancer that is sensitive to an oncolipid-targeting therapy, comprising the steps of: (a) determining the expression levels of ADCK3 and ADCK4 in a biological sample from the subject; (b) identifying the subject as having a cancer that is sensitive to an oncolipid-targeting therapy, if the level of ADCK3 determined in step (a) is significantly higher than a first predetermined reference, and the level of ADCK4 determined in step (a) is significantly lower than a second predetermined reference; and (c) treating the subject identified in step (b) as having a cancer sensitive to an oncolipid-targeting therapy with the oncolipid-targeting therapy.

A further embodiment of the present disclosure is a method for modulating coenzyme Q₁₀ (CoQ₁₀) level in a subject, comprising: (a) determining a baseline CoQ₁₀ level in the subject; (b) administering to the subject an effective amount of an ADCK3 inhibitor; and (c) determining whether the baseline CoQ₁₀ level in the subject has changed.

### BRIEF DESCRIPTION OF THE DRAWINGS

FIGS. 1A-1B show that ADCK3 is the most commonly amplified CoO biosynthesis gene in breast cancer.

FIG. 1A shows the gene alteration data for CoQ₁₀ biosynthesis enzymes in breast cancer. cBioPortal case set: METABRIC, Nature 2012 & Nat Commun 2016 (1904 patients/samples). COQ8A (ADCK3; top panel) is most commonly amplified (28% of study cases).

FIG. 1B shows the gene alteration data for COQ8A (ADCK3), ErbB2 (HER2), estrogen receptors (ESR1, ESR2) and progesterone receptor (PGR) in breast cancer. cBioPortal case set: METABRIC, Nature 2012 & Nat Commun 2016 (2173 patients/samples; mutation and CNA data). The frame marks triple-negative cases which are the majority of ADCK3-amplified cases.

FIGS. 2A-2E show that ferroptosis-sensitive breast cancer cells express high levels of ADCK3.

FIG. 2A shows the ADCK3 gene and transcript expression data taken from the CCLE database. Cells classified as “high-ADCK3” (orange) or “low-ADCK3” (green) based on protein levels shown in FIG. 2B.

FIG. 2B shows the ADCK3 protein levels measured by western blot. For each cell line, ADCK3 levels were normalized to GAPDH levels.

FIG. 2C shows the CoQ₁₀ abundance in 2 million cells of representative cell lines, measured by LC/MS.

FIGS. 2D-2E show that high sensitivity to ferroptosis induction correlated with increased expression of ADCK3. Breast cancer cell lines were treated with ferroptosis inducers FIN56 (FIG. 2D) or RSL3 (FIG. 2E) in 2-fold dilution series for 72 hours. Cell viability was measured with CellTiter-Glo^{®}. Ferroptosis-sensitive breast cancer cells are categorized as ‘high-ADCK3’ expressing (orange), while cells that are less sensitive to ferroptosis induction were the ones with ‘low-ADCK3’ (green).

FIGS. 3A-3E show that genetic inhibition of ADCK3 sensitized for ferroptosis induction in ADCK3-amplified cells.

FIG. 3A shows the CoQ₁₀ abundance upon siRNA-mediated knockdown of ADCK3.

FIG. 3B shows that SKBR3 cells were transfected with siRNA targeting ADCK3 or non-targeting control. At 48 h after transfection, cells were treated with RSL3 in 2-fold dilution series for 48 h, in combination with ferrostatin-1 or idebenone. Viability was measured with CellTiter-Glo^{®}.

FIG. 3C shows the ADCK3 protein levels in CRISPR-mediated ADCK3 knockout SKBR3 cells, compared to GFP-targeting control.

FIG. 3D shows the CoQ10 abundance in CRISPR-mediated ADCK3 knockout SKBR3 cells, vs. GFP-targeting control, measured by LC/MS.

FIG. 3E shows the sensitivity to FIN-56-induced ferroptosis in CRISPR-mediated ADCK3 knockout SKBR3 cells, vs. GFP-targeting control cells. Stable CRISPR cells were treated with FIN56 in 2-fold dilution series in combination with ferrostatin-1, for 48 h. Viability was measured with CellTiter-Glo^{®}.

FIGS. 4A-4D show that shRNA-mediated genetic inhibition of ADCK3 reduced proliferation rate and sensitized ADCK3-amplified cells to ferroptosis induction.

FIG. 4A shows that SKBR3 cells were infected with two different ADCK3-targeting shRNA virions (shADCK3(2) or shADCK3(3)), or with non-targeting control (shCont.) and maintained under puromycin selection. Knockdown was validated by western blot (normalized to GAPDH).

FIG. 4B shows that stable shRNA expressing cells were seeded in 384-well plates at 1000 cells/well. Viability was measured every 24 h for 5 days after seeding, with CellTiter-Glo^{®}.

FIG. 4C shows that shADCK3(2) (orange) and shCont. (grey) cells were treated with RSL3 or FIN56 in 2-fold dilution series in combination with 2 µM ferrostatin-1 for 48 h. Viability was measured with CellTiter-Glo^{®}.

FIG. 4D shows that shADCK3(3) (green) and shCont. (grey) cells were treated with RSL3 or FIN56 in 2-fold dilution series in combination with 2 µM ferrostatin-1 for 48 h. Viability was measured with CellTiter-Glo^{®}.

FIGS. 5A-5C show that overexpression of ADCK3 decreased the sensitivity of SKBR3 cells to ferroptosis induction. ADCK3-FLAG was stably overexpressed in SKBR3 cells through retroviral infection.

FIG. 5A shows that overexpressed ADCK3-FLAF co-localized to the mitochondria similarly to endogenous ADCK3. ADCK3 was stained with anti-ADCK3 antibody and mitochondria was labeled with MitoTracker fluorescent probe. Images are at 100X magnification, bar: 5 µm.

FIG. 5B shows that SKBR3 cells, stably overexpressing ADCK3-FLAG or control vector, were treated with 10 or 20 nM RSL3 for 48 h in the presence or absence of ferrostatin-1. Viability was measured with CellTiter-Glo^{®}.

FIG. 5C shows that SKBR3 cells, stably overexpressing ADCK3-FLAG or control vector, were treated with 78 or 156 nM FIN56 for 48 h in the presence or absence of ferrostatin-1. Viability was measured with CellTiter-Glo^{®}.

FIGS. 6A-6E show the identification of SGC-GAK-1, an ADCK3 inhibitor that potentiated ADCK3-amplified cells to ferroptosis.

FIG. 6A shows that SGC-GAK-1 induced a reduction in ADCK3 protein levels. SKBR3 cells were treated with 50 nM SGC-GAK-1 or vehicle, for 48 h, and analyzed by western blot for ADCK3 and GAPDH levels.

FIG. 6B shows that SGC-GAK-1 induced a reduction in CoQ₁₀ abundance. CoQ₁₀ abundance in SKBR3 cells treated as in FIG. 6A, measured by LC/MS.

FIG. 6C shows that SGC-GAK-1 induced ferroptotic death, rescued by 1 µM ferrostatin-1, in SKBR3 cells in a dose-dependent manner.

FIG. 6D shows that SGC-GAK-1 treatment (as in FIG. 6A) induced an increase in cellular lipid-ROS that was rescued by the addition of 2 µM ferrostatin-1, measured by C11-BODIPY analyzed by flow cytometry.

FIG. 6E shows that SGC-GAK-1 increased the sensitivity of SKBR3 cells to RSL3.

FIG. 7 is a schematic representation of predicted binding of SGC-GAK-1 to the ATP-binding pocket of ADCK3.

FIG. 8 shows the structure of SGC-GAK-1 and its binding affinity for ADCK3.

### DETAILED DESCRIPTION OF THE DISCLOSURE

It is hypothesized that many oncogenic mutations drive addiction to specific oncolipids, analogous to how oncogenic mutations such as in IDH1 cause accumulation of the oncometabolite 2-hydroxyglutarate (2-HG). Discovery of such metabolic dependencies and biomarkers holds the potential to leverage rewired cancer metabolism into more precisely targeted medicines. The present disclosure relates to approaches that target the CoQ₁₀ lipid biosynthesis pathway to eliminate a subset of cancers that are addicted to this potential oncolipid. The present disclosure identified a new druggable breast cancer dependency that may replace current treatment regimens with ones that are more effective and less toxic, to benefit patient survival. More broadly, discovery and targeting of oncolipids will allow for exploiting a new type of cancer dependency.

Coenzyme Q₁₀ (CoQ₁₀; ubiquinone) is a lipophilic molecule synthesized de novo. CoQ₁₀ is present in most membranes of most cell types, and is abundant in mitochondria (Turunen et al. 2004). The ability of this lipid to sustain continuous cycles of oxidation-reduction is the basis of its essential cellular function. While CoQ₁₀ was originally described as a necessary component of the mitochondrial respiratory chain (Mitchell, 1975), another important function of this lipid has become the focus of extensive research in the past decade (Echtay et al. 2000; Fontaine et al. 1998; Hildebrandt and Grieshaber, 2008). Specifically, CoQ₁₀ is the only endogenously synthesized antioxidant that prevents the harmful oxidation of lipids (Bentinger et al. 2007; Ernster and Dallner, 1995). Moreover, in addition to its direct antioxidant activity, CoQ₁₀ contributes to regeneration of other antioxidants, such as the vitamins ascorbate and α-tocopherol (vitamin E) (Villalba and Navas, 2000).

Ferroptosis is an iron-dependent regulated form of oxidative cell death caused by the accumulation of peroxidized PUFA-containing phospholipids (Dixon et al. 2012; Yang and Stockwell, 2016). This form of cell death is controlled by genes and pathways that are distinct and non-overlapping with those that control other regulated cell death mechanisms, such as apoptosis and necroptosis (Dixon et al. 2012; Pasparakis and Vandenabeele, 2015). Ferroptosis is driven by the loss of activity of the lipid repair enzyme glutathione peroxidase 4 (GPX4) (Yang et al. 2014) and by depletion of the intracellular cysteine pool, which is a precursor of glutathione synthesis, caused by inhibition of the system x_{c}⁻ antiporter, which is responsible for cystine uptake (Dixon et al. 2012). Recently, a new mechanism was defined for triggering ferroptosis by the compound FIN56: FIN56 induces depletion of mevalonate-derived CoQ₁₀, an endogenous inhibitor of ferroptosis, through dysregulation of lipid metabolism (Shimada et al. 2016). This suggested that CoQ10-dependent cancers could be selectively targeted for induction of ferroptosis.

Increased ROS and altered redox status typify malignant cells. Indeed, various cancer cell lines have been shown to have altered mitochondria and increased ROS compared to normal cells, making them more vulnerable to ROS (Burdon, 1995; Pelicano et al. 2004; Szatrowski and Nathan, 1991; Tomasetti et al. 2015) and ferroptotic (Toyokuni et al. 2017) cell death. These cancer-associated properties have been suggested to be of therapeutic benefit (Fang et al. 2007; Trachootham et al. 2009). While CoQ₁₀ was initially suggested to contribute to clearance of malignant cells (Lockwood et al. 1994) and to protect from doxorubicin cardiotoxicity (Chen et al. 2017), growing evidence suggests that CoQ₁₀ encompasses significant roles in protecting cancer cells from a tumor-suppressive cell death mechanism, thereby contributing to tumor survival (Papucci et al. 2003; Brea-Calvo et al. 2006). These protective attributes of CoQ₁₀ in tumor cells may counteract chemotherapeutics and mark it as a harmful dietary supplement for cancer patients. Supporting this idea are recent clinical studies showing that antioxidant dietary supplements, such as vitamin E (which also protects against ferroptotic death (Shimada et al. 2016)), increase the risk for cancer, as well as cancer recurrence, and increases overall patient mortality, especially amongst smokers who are more prone to oxidative damage (Harvie, 2014). This suggests that some tumors that would otherwise be eliminated through oxidative death thrive in the presence of antioxidants. Additionally, most chemotherapeutic drugs, such as camptothecin, doxorubicin, and methotrexate, do not provoke any decrease in antioxidants. Instead, they frequently induce a compensating increase in antioxidant defenses as a protective mechanism against ROS, leading to drug resistance (Brea-Calvo et al. 2006).

Accordingly, it is hypothesized that some cancers overcome tumor-suppressing ferroptotic cell death by becoming addicted to increased production of CoQ₁₀. Supporting the increased sensitivity of cancer cells to ferroptotic death are the observations that depriving many cancer cell lines of cysteine, selenium or NADPH (needed for glutathione peroxidase 4, which counteracts lipid peroxidation) results in ferroptosis (Dixon et al. 2012; Yang et al. 2014; Dixon et al. 2014; Hayano et al. 2016; Skouta et al. 2014), and that wild-type p53 and BAP1 exert tumor suppressive activity through downregulating system x_{c}⁻, leading to ferroptosis (Jiang et al. 2015). Additionally, ferroptosis-protective modulators are commonly upregulated in many cancers (system x_{c}⁻ (Ishimoto et al. 2011; Ogunrinu et al. 2010), GPX4 (Yang et al. 2014; Guerriero et al. 2015), NADPH and NRF2 (Wu et al. 2011)). The addiction to ferroptosis-inhibiting mechanisms is exemplified by the acceleration of lung cancer upon administration of the lipophilic antioxidant vitamin E (Sayin et al. 2014), and the abundance of oncogenic mutations that drive the mevalonate biosynthesis pathway (Freed-Pastor et al. 2012; Gruenbacher and Thurnher, 2015; Jiang et al. 2014), leading to increased production of CoQ₁₀. Moreover, targeting CoQ₁₀ has cytotoxic effects on lung cancers (Ortiz et al. 2017); conversely, several chemotherapeutic drugs induce an increase in CoQ₁₀, contributing to cell survival and chemotherapy resistance to these conventional therapeutics (Brea-Calvo et al. 2006).

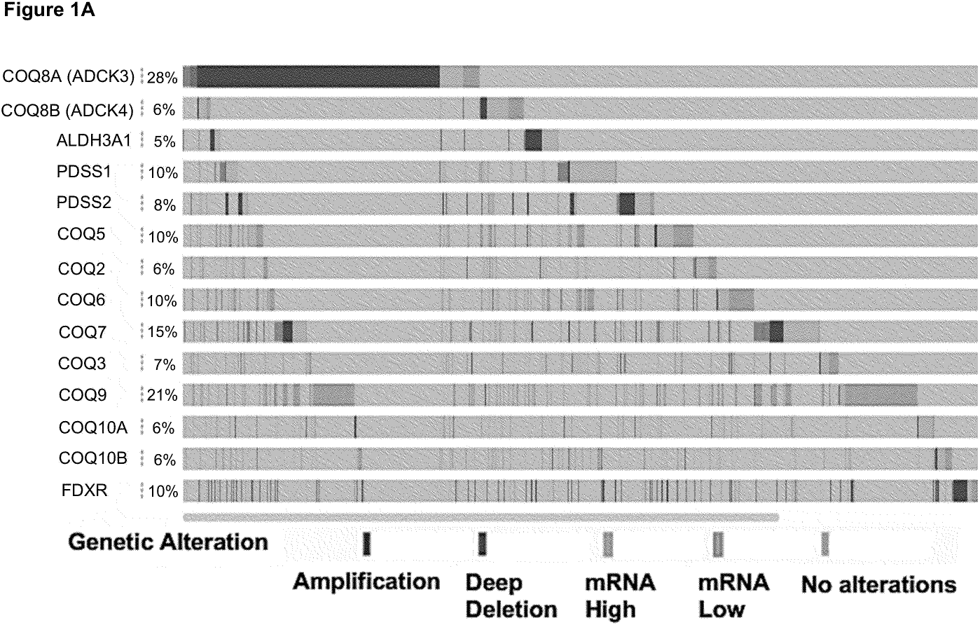

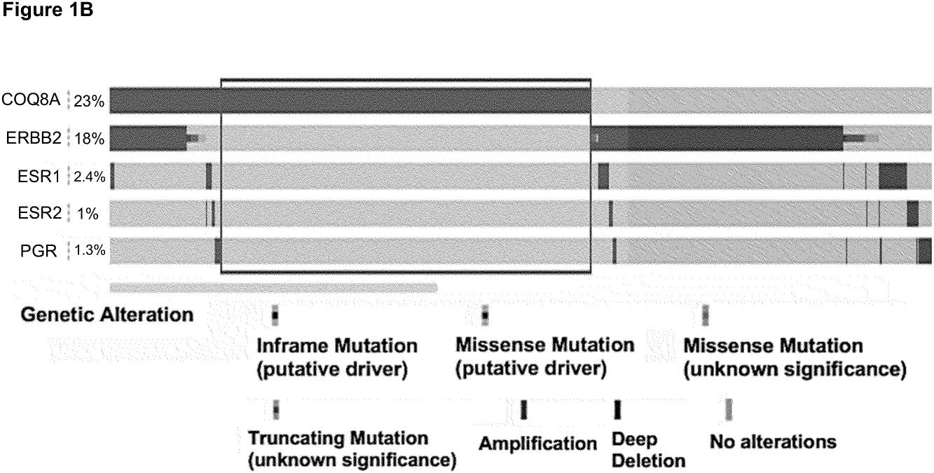

The present disclosure provides an approach that targets CoQ₁₀ biosynthesis to selectively induce ferroptotic cell death in cancer cells that are addicted to increased production of CoQ₁₀. CoQ₁₀ biosynthesis involves 14 kinases and regulatory proteins (Acosta et al. 2016; Stefely and Pagliarini, 2017). While systemic depletion of CoQ₁₀ may cause toxicity, CoQ₁₀ biosynthesis is differentially regulated in many cancer cells, and genes associated with CoQ₁₀ biosynthesis pathway are amplified or mutated in diverse cancers (see cBioPortal (Cerami et al. 2012; Gao et al. 2013)). Among the human proteins known to participate in CoQ₁₀ biosynthesis, ADCK3 (COQ8A), a kinase that has a regulatory role in CoQ₁₀ biosynthesis (Poon et al. 2000; Stefely et al. 2015), is unambiguously amplified in many cancers (cBioPortal (Cerami et al. 2012; Gao et al. 2013)), and most abundantly in breast cancers, suggesting addiction to ADCK3 in these contexts. Importantly, in breast cancer patients, ADCK3 gene amplification is the most common amplification amongst all of the known CoQ₁₀ biosynthesis genes (Stefely and Pagliarini, 2017) (see FIG. 1A and cBioPortal (Cerami et al. 2012; Gao et al. 2013)). Additionally, ADCK3-amplified breast cancers are commonly triple-negative (FIG. 1B and cBioPortal (Cerami et al. 2012; Gao et al. 2013)), which are considered more difficult-to-treat subtypes due to the lack of a druggable precision target. Of note, induction of ferroptotic death was recently shown to be effective for breast cancer treatment (Hasegawa et al. 2016; Timmerman et al. 2013), and mesenchymal breast cancers, associated with resistance to multiple treatment modalities, were shown to be more susceptible to induction of ferroptosis (Viswanathan et al. 2017). This suggests that enhancing sensitivity to ferroptosis induction through targeting ADCK3 offers a promising treatment opportunity for difficult-to-treat breast cancers.

Reduction of CoQ₁₀ levels through inhibition of ADCK3 entails a wide therapeutic window for cancer therapeutics. The closest homolog of ADCK3 is ADCK4 (COQ8B), which can compensate for ADCK3 in normal tissues, but is deleted or underexpressed in many cancers. Thus, cancers that primarily use ADCK3 over ADCK4 to enhance CoQ₁₀ biosynthesis will be susceptible to selective triggering of cell death by ADCK3 inhibition, which would not deplete CoQ₁₀ in normal tissues that make use of ADCK4. Additionally, ADCK3 mutation or deletion is associated with only mild CNS phenotypes in humans and mice (Horvath et al. 2012; Mollet et al. 2008; Stefely et al. 2016). This suggests that ADCK3 inhibitors that do not penetrate the blood-brain barrier may have a high therapeutic index for non-CNS cancers addicted to increased production of CoQ₁₀ through ADCK3 amplification. Moreover, statins that deplete CoQ₁₀ are generally well tolerated with rare exceptions, supporting the hypothesis that ADCK3 inhibitors will have low toxicity in normal (non-CoQ₁₀-addicted) cells. The lack of effect for statins on breast cancer risk can be explained by the known biodistribution of these drugs, primary localized to the liver (Stancu and Sima, 2001). In addition, ADCK3 is also amplified or overexpressed in a smaller percentage of other cancers, suggesting that a genetically-targeted patient population can be defined beyond breast cancers.

Accordingly, one aspect of the present disclosure is targeting an underexplored tumor dependency - the addiction to increased biosynthesis of the lipophilic antioxidant CoQ₁₀. Increased generation of ROS and altered redox status are known to typify malignant cells; yet, targeting oncogenic lipids, such as CoQ₁₀, that enable tumor cells to thrive upon increased ROS conditions is an underexplored therapeutic avenue. This disclosure is, inter alia, focused on targeting the addiction of a subset of breast cancer cells to increased biosynthesis of CoQ₁₀, in order to induce ferroptosis. The vast majority of current breast cancer treatments induce apoptotic cell death pathways. Although inducing apoptosis has been shown to be clinically effective in breast cancer subtypes, recurrence and resistance to treatment are still major problems in breast cancer treatment.

Another aspect of the present disclosure is a novel approach to targeted therapy that complements apoptosis induction - the induction of ferroptosis. This can be a novel avenue to treat breast cancer subtypes that are currently considered difficult-to-treat, by inducing ferroptotic death through targeting metabolic dependencies.

Accordingly, one embodiment of the present disclosure is a method for treating or ameliorating the effects of a disorder in a subject, comprising administering to the subject an effective amount of an agent that increases lipid-based reactive oxygen species (ROS).

As used herein, the term “reactive oxygen species” or “ROS” means chemically reactive molecules, such as free radicals, containing oxygen. Non-limiting examples of ROS include peroxides, superoxide, hydroxyl radical, singlet oxygen and alpha-oxygen.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

In some embodiments, the disorder is associated with accumulation of an oncolipid. In some embodiments, the oncolipid is an antioxidant. In some embodiments, the antioxidant is endogenous to the subject. In some embodiments, the antioxidant is coenzyme Q₁₀ (CoQ₁₀).

In some embodiments, the disorder is a cancer. In some embodiments, the cancer is selected from the group consisting of head and neck cancer, prostate cancer, stomach cancer, colorectal cancer, bladder cancer, thymoma, thymic carcinoma, lung adenocarcinoma, uterine carcinosarcoma, cervical carcinosarcoma, esophageal carcinosarcoma, non-small-cell lung carcinoma (NSCLC), pancreatic cancer, breast cancer, melanoma, diffuse large B-cell lymphoma (DLBCL), ovarian cancer, liver cancer, chronic lymphocytic leukemia (CLL), cholangiocarcinoma, neuroendocrine prostate cancer (NEPC), and combinations thereof.

In some embodiments, the disorder is breast cancer. In some embodiments, the breast cancer is mesenchymal breast cancer. In some embodiments, the breast cancer is triple-negative breast cancer. In some embodiments, the breast cancer is unresectable.

In the context of the present disclosure, “mesenchymal” refers to a state of tumor progression, characterized by loosely associated cells and disorganized cellular layers that lack polarity and tight cell-to-cell adhesion proteins. Such morphology of mesenchymal cells is better adapted to cell migration. A mesenchymal cancer can either be mesenchymal origin (e.g., sarcomas) or epithelial origin (e.g., breast cancer) but at the end or late stage of epithelial-mesenchymal transition (EMT) that is typically characterized as loss of epithelial cell adhesion protein E-cadherin and cytokeratins together with the gain of mesenchymal-associated molecules N-cadherin, Vimentin, and fibronectin. Exemplary EMT-related biomarkers include Vimentin, N-cadherin, Snail, Slug, Twist, N-cadherin and cytokeratins expression.

In some embodiments, the subject is a mammal. In some embodiments, the mammal is selected from the group consisting of humans, veterinary animals, and agricultural animals. In some embodiments, the subject is a human.

In some embodiments, the disorder is associated with overexpression of ADCK3. In some embodiments, the agent increases lipid-based reactive oxygen species (ROS) by inhibiting coenzyme Q₁₀ (CoQ₁₀) production. In some embodiments, the agent is an ADCK3 inhibitor. In the present disclosure, any ADCK3 inhibitor may be used so long as it is safe and effective for the subject. Accordingly, in some embodiments, the ADCK3 inhibitor is selected from dasatinib, PD-173955, R406, TG-100-115, UNC-CA157, SGC-GAK-1, pharmaceutical compositions thereof and combinations thereof. In some embodiments, the ADCK3 inhibitor is SGC-GAK-1 or a pharmaceutical composition thereof.

In some embodiments, the method disclosed herein further comprises coadministering to the subject an effective amount of a ferroptosis inducer. In some embodiments, the ferroptosis inducer is selected from the group consisting of erastin, imidazole ketone erastin (IKE), piperazine erastin (PE), sulfasalazine, sorafenib, RSL3, ferroptosis inducer 56 (FIN56), caspase-independent lethal 56 (CIL56), ferroptosis inducer endoperoxide (FINO₂), pharmaceutical compositions thereof and combinations thereof.

As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.) In the context of this disclosure, a therapy based on other non-ferroptosis cell death such as apoptosis can be coadministered to the subject.

In some embodiments, the method disclosed herein further comprises coadministering to the subject a therapy selected from the group consisting of surgery, chemotherapy, radiation therapy, immunotherapy, and combinations thereof.

In some embodiments, the chemotherapy comprises administering to the subject a therapeutically useful chemotherapeutic agent. Such an agent may, for example, be selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, pharmaceutical compositions thereof and combinations thereof.

In some embodiments, the immunotherapy comprises administering to the subject a therapeutically useful immunotherapeutic agent. In the present disclosure, such an agent may include chimeric antigen receptor (CAR) T-cell therapeutics, T-cell receptor (TCR) therapeutics, tumor-infiltrating lymphocyte (TIL) therapeutics, monoclonal antibody therapeutics, immune checkpoint inhibitors and combinations thereof. For example, the immunotherapeutic agent may be selected from the group consisting of ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab, pharmaceutical compositions thereof and combinations thereof.

Another embodiment of the present disclosure is a method for treating a subject with a cancer that is sensitive to an oncolipid-targeting therapy, comprising the steps of: (a) determining the expression levels of ADCK3 and ADCK4 in a biological sample from the subject; (b) identifying the subject as having a cancer that is sensitive to an oncolipid-targeting therapy, if the level of ADCK3 determined in step (a) is significantly higher than a first predetermined reference, and the level of ADCK4 determined in step (a) is significantly lower than a second predetermined reference; and (c) treating the subject identified in step (b) as having a cancer sensitive to an oncolipid-targeting therapy with the oncolipid-targeting therapy.

In the context of this disclosure, the biological sample can be a tissue section, a biopsy, blood, or other appropriate bodily fluid. In some embodiments, the biological sample is obtained from the cancerous tissue of the subject.

In some embodiments, the first predetermined reference is the expression level of ADCK3 in normal tissue of the subject, and the second predetermined reference is the expression level of ADCK4 in normal tissue of the subject. In some embodiments it is contemplated that any conventional method for determining the expression level of a protein or fragment thereof may be used to determine the levels of ADCK3 and ADCK4, including, e.g., the methods disclosed in the examples below.

In some embodiments, the oncolipid is coenzyme Q₁₀ (CoQ₁₀).

In some embodiments, the oncolipid-targeting therapy comprises administering to the subject an effective amount of an ADCK3 inhibitor as defined herein. In some embodiments, the ADCK3 inhibitor is selected from dasatinib, PD-173955, R406, TG-100-115, UNC-CA157, SGC-GAK-1, pharmaceutical compositions thereof and combinations thereof. In some embodiments, the ADCK3 inhibitor is SGC-GAK-1 or a pharmaceutical composition thereof.

In some embodiments, the cancer is selected from the group consisting of head and neck cancer, prostate cancer, stomach cancer, colorectal cancer, bladder cancer, thymoma, thymic carcinoma, lung adenocarcinoma, uterine carcinosarcoma, cervical carcinosarcoma, esophageal carcinosarcoma, non-small-cell lung carcinoma (NSCLC), pancreatic cancer, breast cancer, melanoma, diffuse large B-cell lymphoma (DLBCL), ovarian cancer, liver cancer, chronic lymphocytic leukemia (CLL), cholangiocarcinoma, neuroendocrine prostate cancer (NEPC), and combinations thereof.

In some embodiments, the cancer is breast cancer. In some embodiments, the breast cancer is mesenchymal breast cancer. In some embodiments, the breast cancer is triple-negative breast cancer. In some embodiments, the breast cancer is unresectable.

In some embodiments, the subject is a mammal. In some embodiments, the mammal is selected from the group consisting of humans, veterinary animals, and agricultural animals. In some embodiments, the subject is a human.

A further embodiment of the present disclosure is a method for modulating coenzyme Q₁₀ (CoQ₁₀) level in a subject, comprising: (a) determining a baseline CoQ₁₀ level in the subject; (b) administering to the subject an effective amount of an ADCK3 inhibitor; and (c) determining whether the baseline CoQ₁₀ level in the subject has changed.

In some embodiments, the ADCK3 inhibitor is as defined herein, such as, e.g., SGC-GAK-1 or a pharmaceutical composition thereof. In some embodiments, a medical professional may use the result of this method to adjust, i.e., to increase, decrease or leave unchanged, how much of the ADCK3 inhibitor is administered to the subject. In some embodiments, a medical professional may use the result of this method to monitor the progression of disease, e.g., cancer, in the subject.

In some embodiments, the CoQ₁₀ levels in the subject are determined by any conventional method known to those of skill in the art, such as, e.g., LC-MS. In some embodiments, the measured CoQ₁₀ levels include reduced, oxidized, and/or total cellular CoQ₁₀ levels.

As used herein, the terms “modulate”, “modulating”, “modulator” and grammatical variations thereof mean to change, such as increasing, decreasing or reducing the abundance of an oncolipid such as CoQ₁₀.

The following examples are provided to further illustrate the methods of the present disclosure. These examples are illustrative only and are not intended to limit the scope of the disclosure in any way.

### EXAMPLES

### Example 1 Ferroptosis-Sensitive Breast Cancer Cell Lines Express High Levels of ADCK3

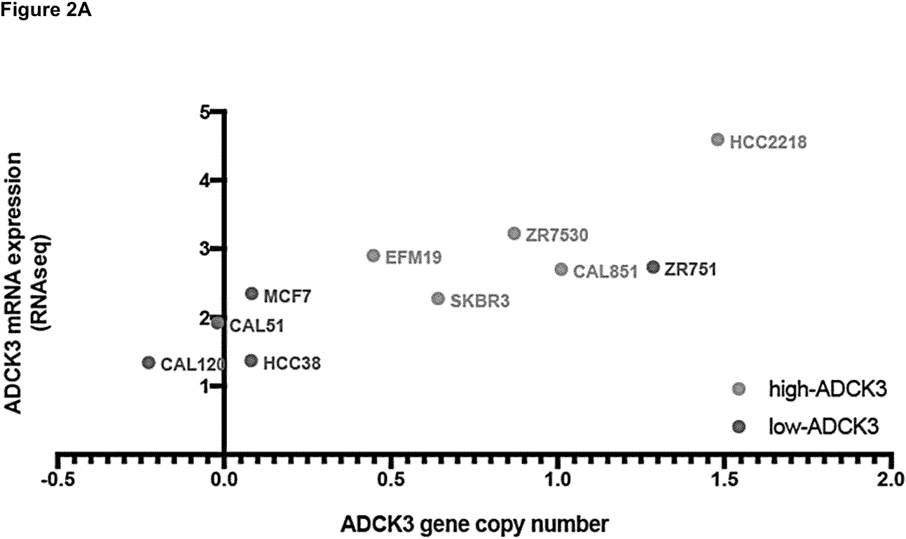

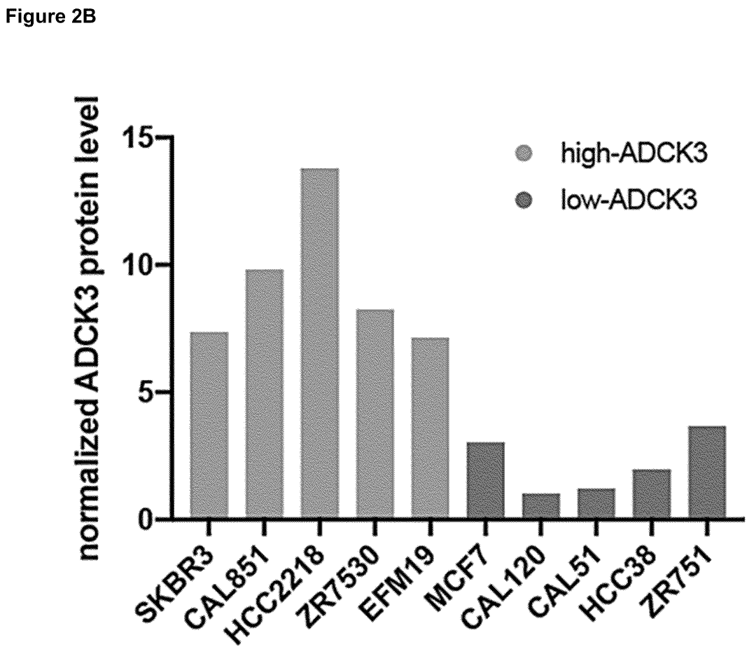

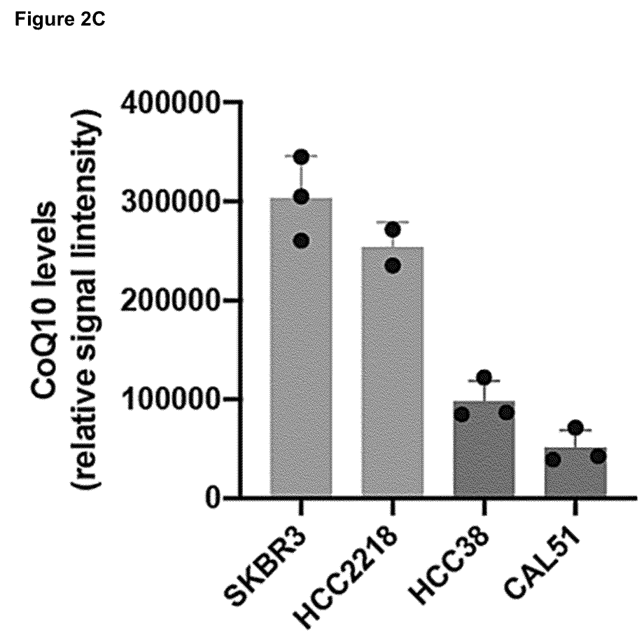

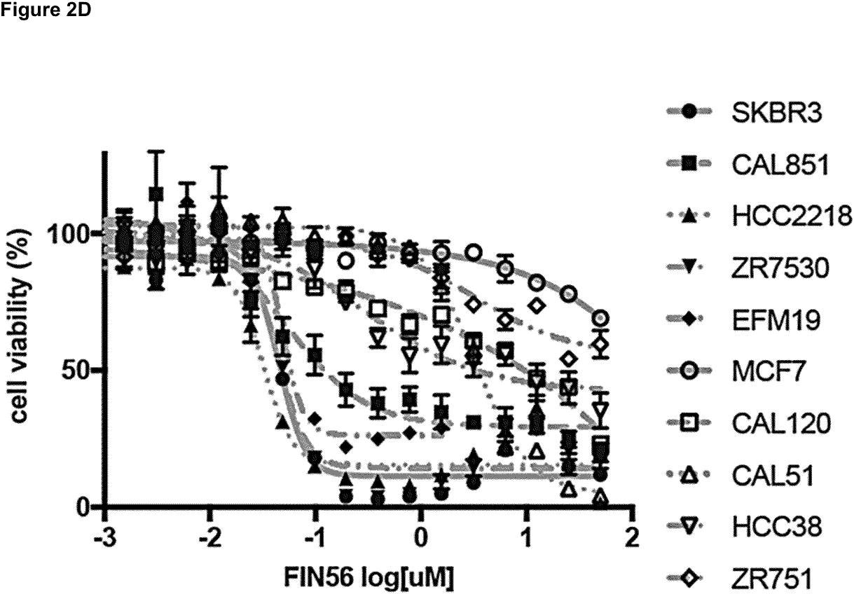

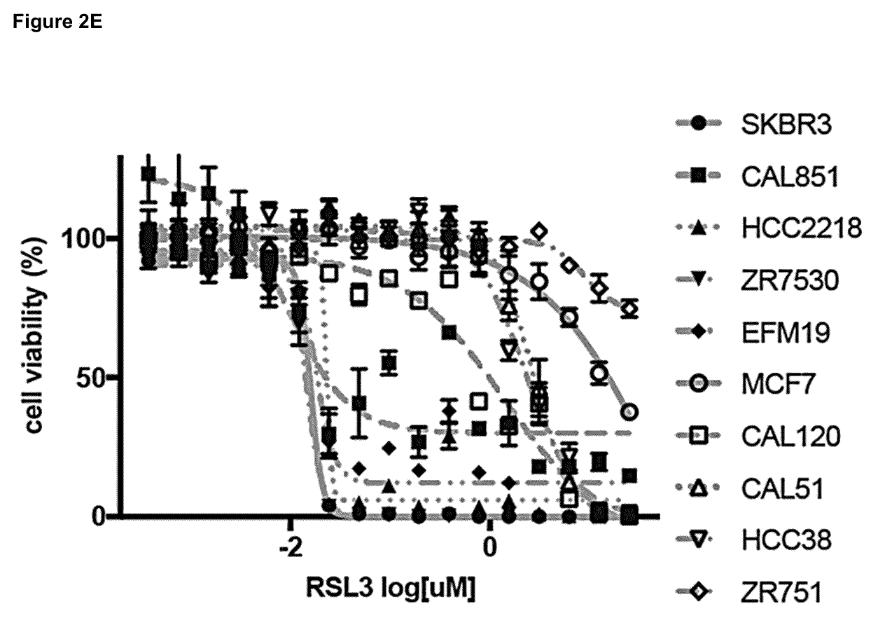

A panel of 10 breast cancer cell lines was selected form the Broad Institute Cancer Cell Line Encyclopedia database (CCLE; https://portals.broadinsitute.org/ccle),based on their ADCK3 copy number and mRNA expression levels, to have a good representation of ADCK3-amplified cells (termed here as ‘high-ADCK3’), as well as a control group expressing normal levels of ADCK3 (termed here as ‘low-ADCK3’; FIG. 2A). To confirm a correlation between high ADCK3 gene/transcript expression and protein expression, ADCK3 levels were measured by western blot (FIG. 2B). Cells were divided into high-ADCK3 (orange in FIGS. 2A-2E) and low-ADCK3 (green in FIGS. 2A-2E) according to the protein expression level. Of note, in the ZR751 cell line, high gene expression (FIG. 2A) did not result in high ADCK3 protein expression (FIG. 2B), and thus was considered to be a low-ADCK3 expressing cell line. High ADCK3 protein levels correlated with high CoQ₁₀ abundance, measured by LC-MS (FIG. 2C). It was hypothesized that hypersensitivity of a subset of breast cancers to ferroptotic death (possibly due to imbalanced redox consumption and signaling and/or iron-rich microenvironment; both of which often typify malignancies (Toyokuni et al. 2017; Hanahan and Weinberg, 2011)) was the driver for the addiction of this subset of ferroptosis-sensitive cells to increased production of CoQ₁₀ for survival (to evade ferroptotic death) through increased ADCK3 activity.

Indeed, breast cancer cells that showed distinct higher sensitivity to ferroptosis induction through two different mechanisms (induced by FIN56 which inhibits CoQ₁₀ biosynthesis and GPX4 activity, and by RSL3 which inhibits GPX4 activity), were the ones with amplified ADCK3 expression (FIGS. 2D-2E). This fit our hypothesis that increased ferroptosis sensitivity was the driver tumor-survival mechanism for upregulation of ADCK3 expression (maintaining increased abundance of CoQ₁₀). Of note, the expression level of ADCK4 is relatively low and similar across all breast cancer cell lines tested (not shown). This suggested that perturbation to CoQ₁₀ biosynthesis through ADCK3 inhibition may selectively sensitize ADCK3-amplified breast cancer subtypes to ferroptosis, while maintaining normal CoQ₁₀ levels in normal tissue due to ADCK4 compensation.

### Example 2 ADCK3 Knockdown Reduced CoQ₁₀ Levels

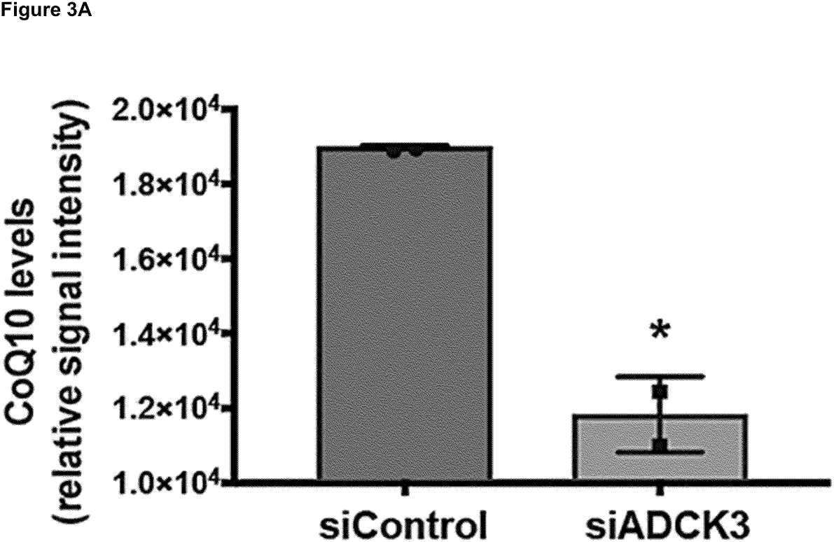

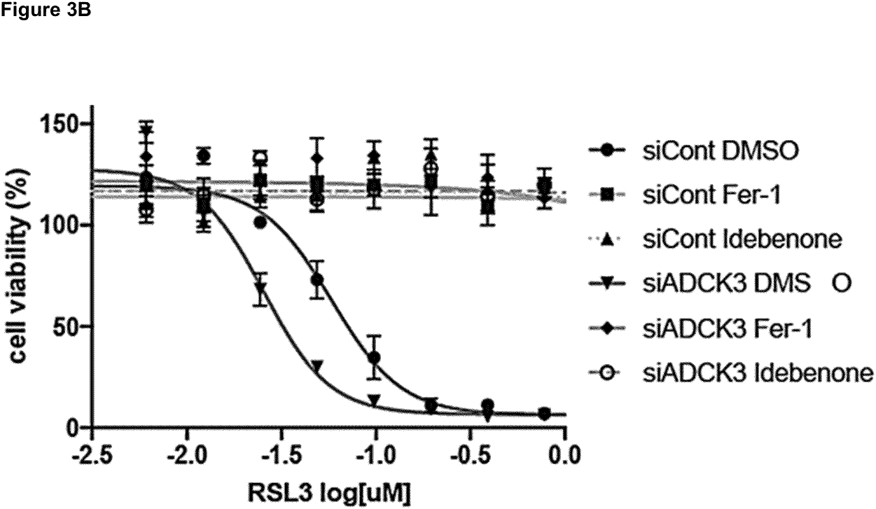

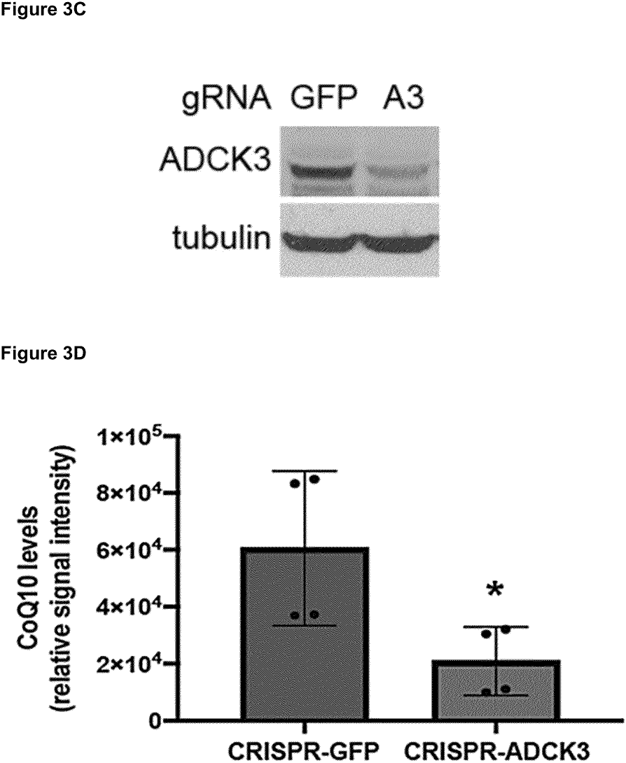

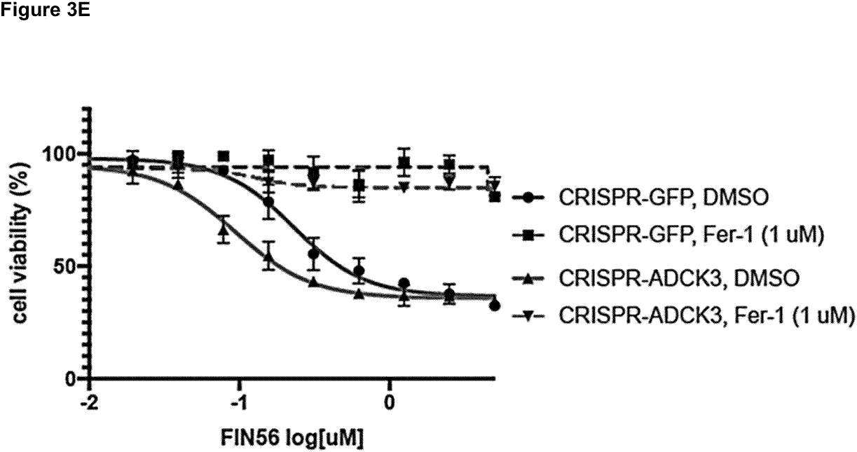

In a high-ADCK3 expressing breast cancer model cell line, SKBR3, siRNA-mediated knockdown of ADCK3 resulted in a significant decrease in the reduced-CoQ₁₀ (ubiquinol; FIG. 3A) cellular pool as well as in the reduced/oxidized CoQ₁₀ ratio (ubiquinol/ubiquinone; not shown). Reduced CoQ₁₀ is the active antioxidant form of CoQ₁₀, potentially protective from ferroptosis. Similar reduction in reduced CoQ10 abundance was measured upon CRISPR-mediated knockout of ADCK3 in the same cell line (FIGS. 3C-3D). These data suggested that ADCK3 contributes to CoQ₁₀ biosynthesis and that inhibition of ADCK3 will impair such biosynthesis in addicted cells.

### Example 3 Genetic Knockdown of ADCK3 Inhibited SKBR3 Cell Growth

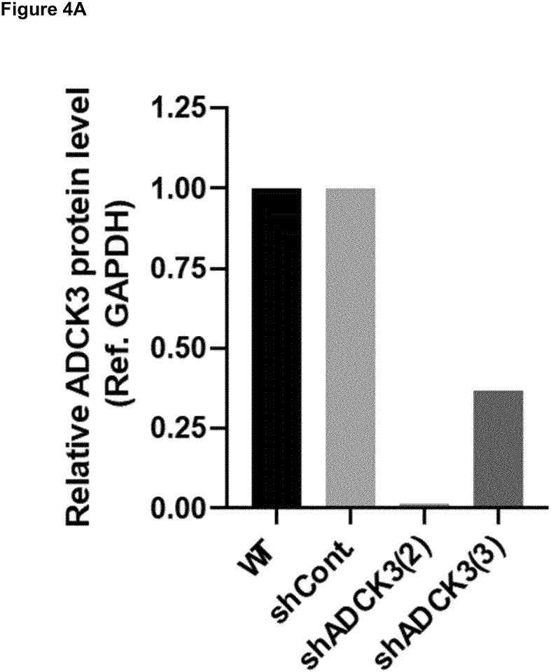

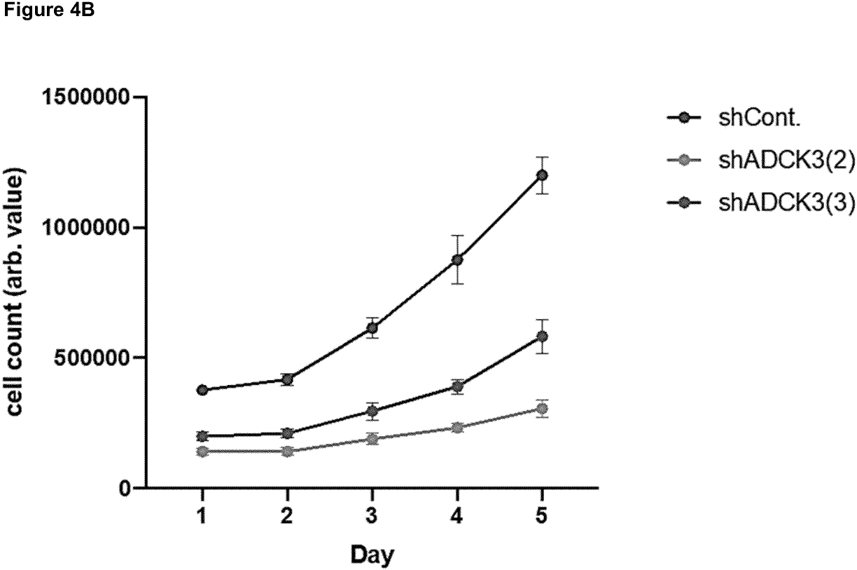

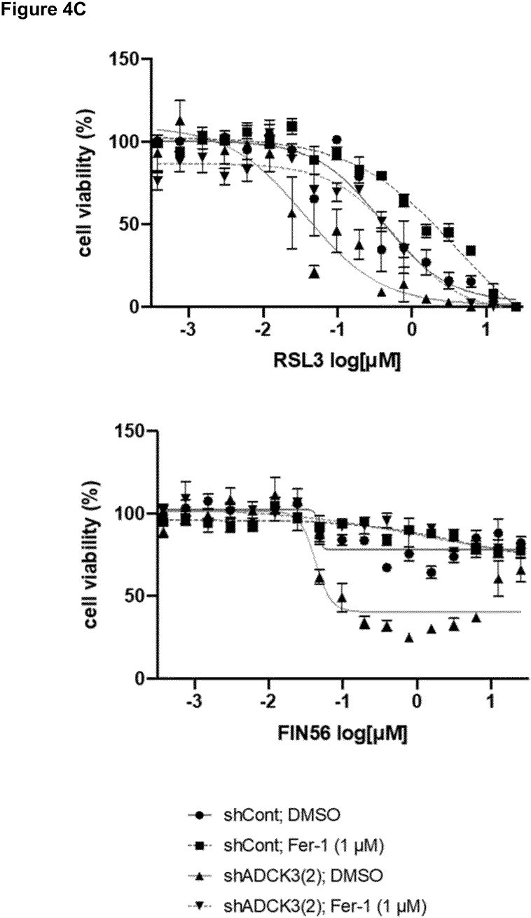

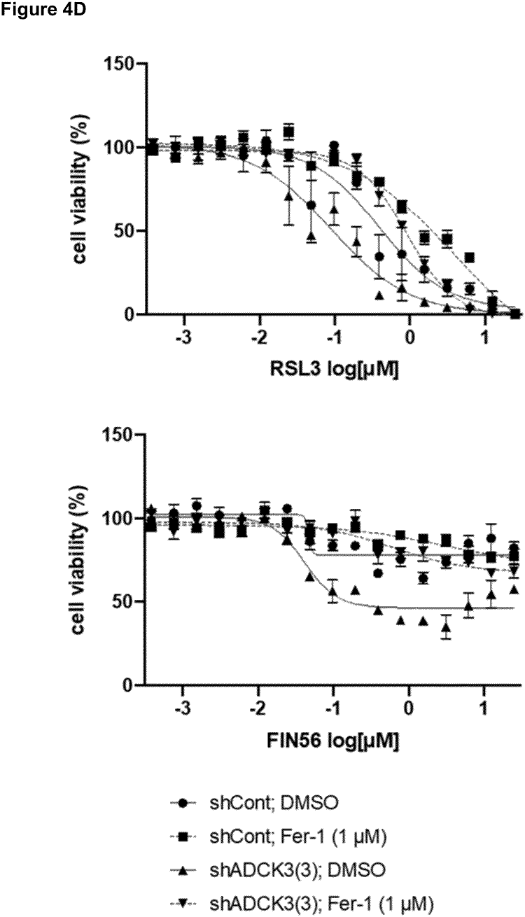

SKBR3 cells infected with two different ADCK3-targeting shRNA expressing viral particles (shADCK3(2) and shADCK3(3); FIG. 4A), presented significantly reduced proliferation rate compared to cells infected with the non-targeting control shRNA virions (FIG. 4B). This shows that high levels of ADCK3, and subsequently of CoQ₁₀, is required for the growth of these cells, implicating for metabolic dependency on the CoQ₁₀ pathway.

### Example 4 Inhibition of CoQ₁₀ Biosynthesis Selectively Sensitized ADCK3-Amplified Breast Cancer Cells to Ferroptosis Induction

It was further demonstrated that genetic perturbation to CoQ₁₀ biosynthesis through inhibition of ADCK3 increased the sensitivity of ADCK3-amplified breast cancer cells to ferroptosis inducers. Transient siRNA-mediated knockdown of ADCK3, which significantly reduced the levels of reduced CoQ₁₀ (FIG. 3A), increased the sensitivity of the knockdown cells to the ferroptosis inducer RSL3 (FIG. 3B). This death was rescued both by the addition of the ferroptosis inhibitor ferrostatin-1 and the CoQ₁₀ water-soluble analog, idebenone, supporting the hypothesis that CoQ₁₀ plays a central role in suppressing ferroptotic death in this subset of cancer cells. Similar ferroptosis-potentiation effect was observed with a stable CRISPR-mediated knockout of ADCK3 treated with FIN56 (FIG. 3E) as well as shRNA-mediated ADCK3 knockout SKBR3 cells, treated with RSL3 or FIN56 (FIGS. 4C-4D). This death and sensitization were also rescued by addition of ferrostatin-1, confirming ferroptosis as the death mechanism.

### Example 5 Overexpression of ADCK3 Reduced the Sensitivity to Ferroptosis Induction

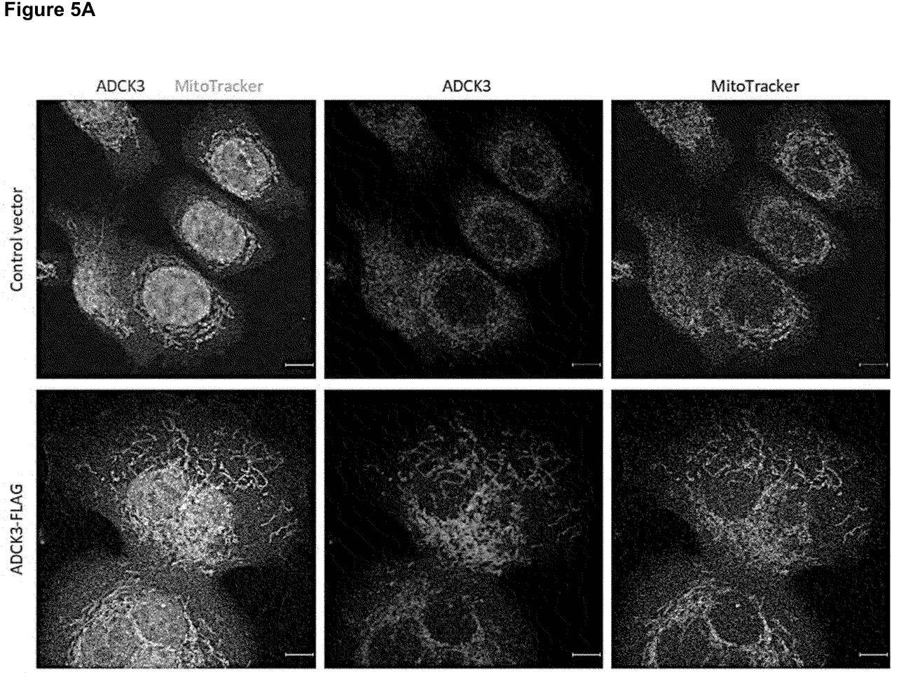

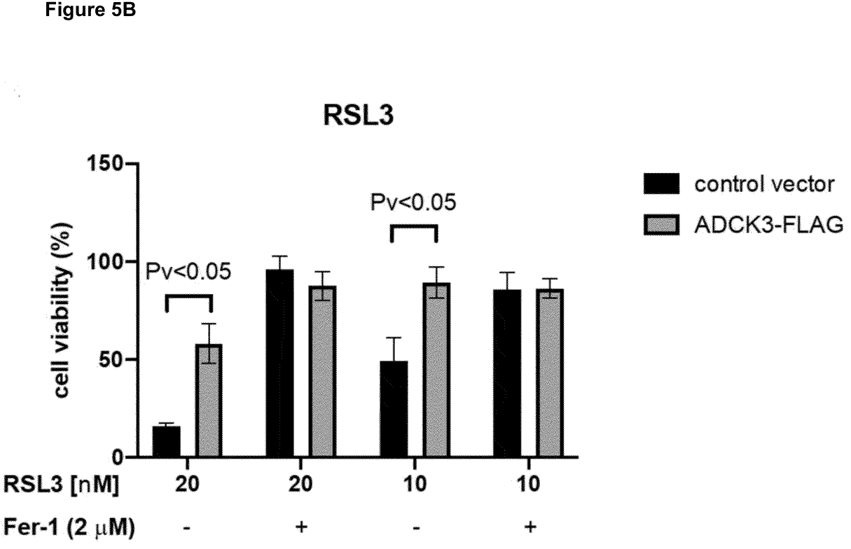

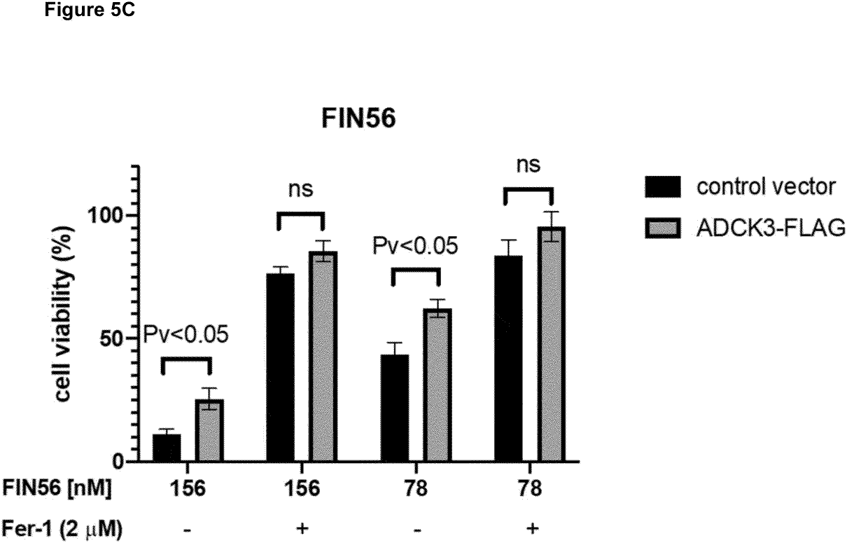

ADCK3-FLAG was stably overexpressed in SKBR3 cells through retroviral infection. First, proper localization of the overexpressed protein to the mitochondria was validated by fluorescent microscopy. Similarly to the localization pattern of endogenous ADCK3 to the mitochondria, overexpressed ADCK3-FLAG co-localized with a MitoTracker fluorescent probe (FIG. 5A). Induction of ferroptotic death with RSL3 (FIG. 5B) or FIN56 (FIG. 5C) resulted in a statistically significant reduction in cytotoxic effect in cells overexpressing ADCK3, compared to control. These results confirm the role of ADCK3 (and CoQ₁₀) in protecting against ferroptotic death in this cellular context.

### Example 6 Identification of a Potential ADCK3 Inhibitor that Induced a Reduction in ADCK3 and CoQ₁₀ Abundance, and Potentiated ADCK3-Amplified Breast Cancer Cells to Ferroptotic Death

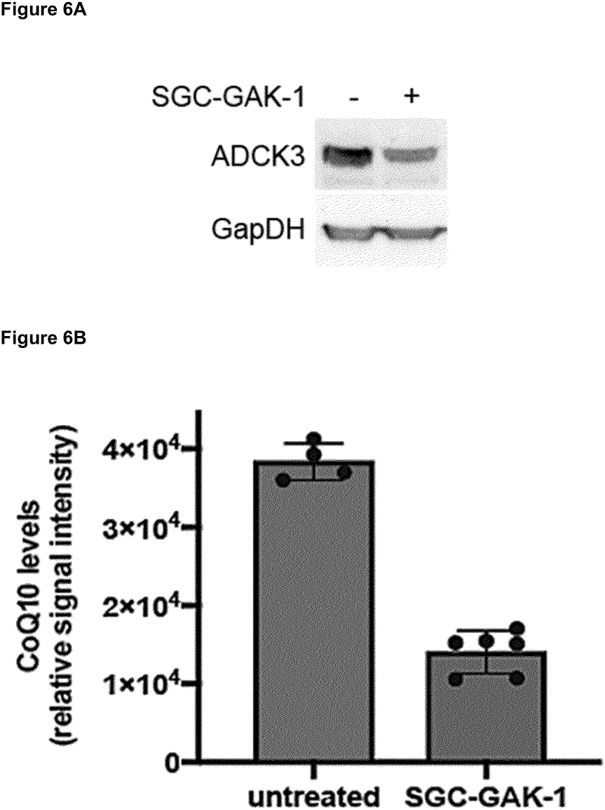

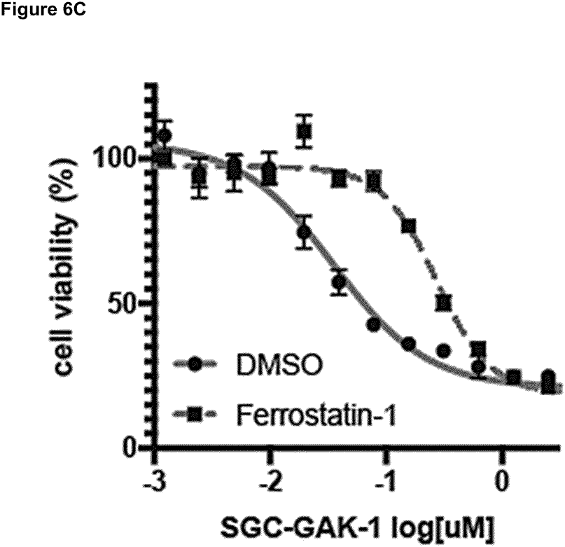

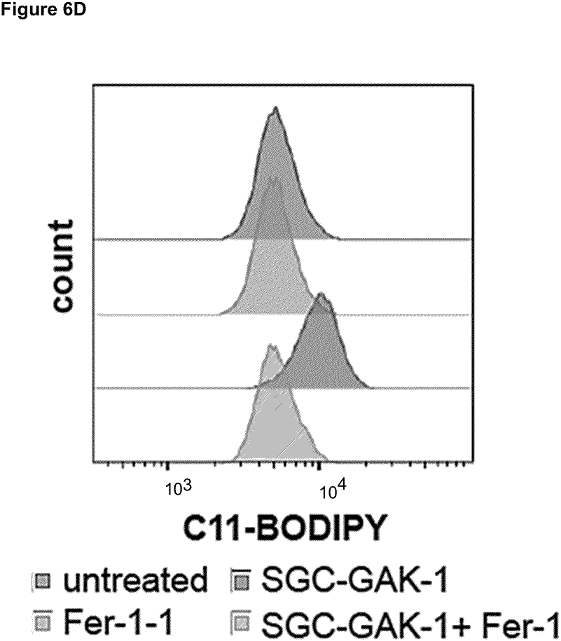

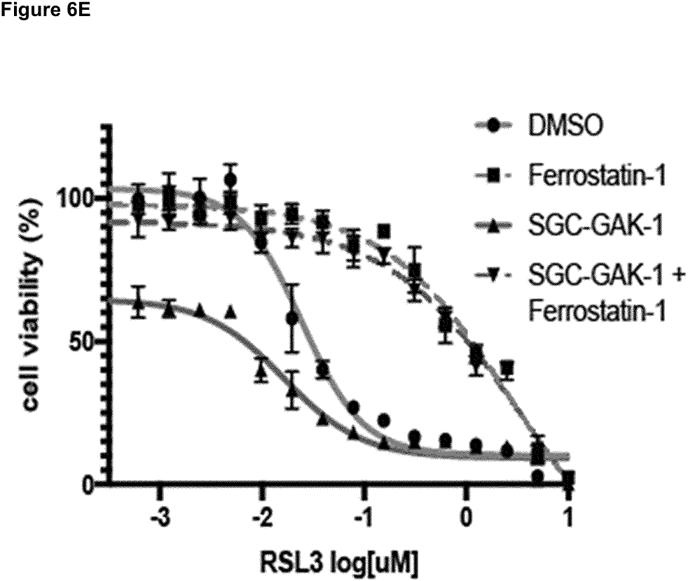

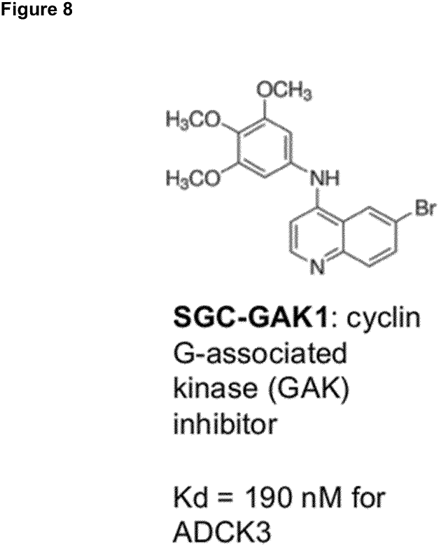

SGC-GAK-1, a cyclin G associated kinase (GAK) inhibitor (FIG. 8), was reported to have a nanomolar inhibitory activity against ADCK3 (Asquith et al. 2019). Treatment of an ADCK3-amplified breast cancer cell line, SKBR3, with 50 nM SGC-GAK-1 for 48 hours resulted in a marked reduction in ADCK3 protein levels (FIG. 6A) and reduced CoQ₁₀ levels (FIG. 6B). This inhibition of ADCK3 is in line with the demonstrated reduction in CoQ₁₀ levels by genetic inhibition of ADCK3 (FIGS. 3A-3E and FIGS. 4A-4D). Moreover, treatment with SGC-GAK-1 alone induced cell death that was rescued by the ferroptosis inhibitor ferrostatin-1 (perhaps through ADCK3 inhibition; FIG. 6C), which was accompanied by an increase in lipid-ROS (measured by C11-BODIPY; FIG. 6D), a hallmark of ferroptosis. Thus, it was concluded that SGC-GAK-1 induces ferroptosis in ADCK3-amplified cells, perhaps by reducing the levels of ADCK3 and CoQ₁₀. SGC-GAK-1 also further potentiated SKBR3 cells to RSL3-mediated ferroptotic death (FIG. 6E), suggesting the potential of this drug to sensitize ferroptosis-sensitive cells when the conditions support such death (typically in a tumor microenvironment).

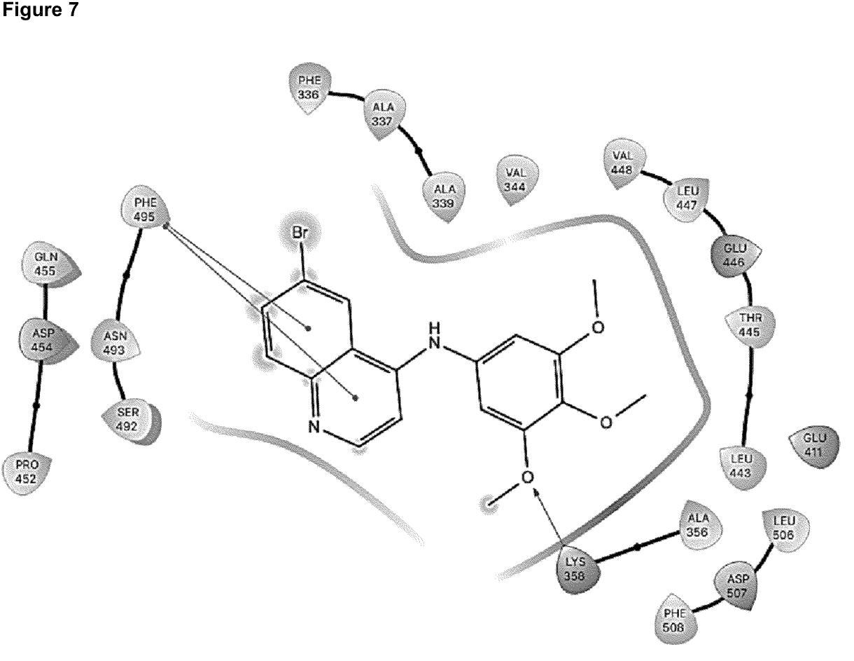

Docking experiments confirmed SGC-GAK-1 binding to ADCK3. Glide docking of SGC-GAK-1 to ADCK3 using Schrodinger Suite modeling software revealed binding interactions in the ATP pocket including tight hinge region binding of the quinoline nitrogen of SGC-GAK-1 to Val448 (FIG. 7). This model defines a pharmacophore that can be used as the basis for analog design and synthesis to improve potency, selectivity, and ADME properties to discover a novel therapeutic drug for ADCK3-amplified breast cancers.

### Documents Cited

Turunen, M., J. Olsson, and G. Dallner, Metabolism and function of coenzyme Q. Biochim Biophys Acta, 2004. 1660(1-2): p. 171-99.

Mitchell, P., Protonmotive redox mechanism of the cytochrome b-c1 complex in the respiratory chain: protonmotive ubiquinone cycle. FEBS Lett, 1975. 56(1): p. 1-6.

Echtay, K.S., E. Winkler, and M. Klingenberg, Coenzyme Q is an obligatory cofactor for uncoupling protein function. Nature, 2000. 408(6812): p. 609-13.

Fontaine, E., F. Ichas, and P. Bernardi, A ubiquinone-binding site regulates the mitochondrial permeability transition pore. J Biol Chem, 1998. 273(40): p. 25734-40.

Hildebrandt, T.M. and M.K. Grieshaber, Three enzymatic activities catalyze the oxidation of sulfide to thiosulfate in mammalian and invertebrate mitochondria. FEBS J, 2008. 275(13): p. 3352-61.

Bentinger, M., K. Brismar, and G. Dallner, The antioxidant role of coenzyme Q. Mitochondrion, 2007. 7 Suppl: p. S41-50.

Ernster, L. and G. Dallner, Biochemical, physiological and medical aspects of ubiquinone function. Biochim Biophys Acta, 1995. 1271 (1): p. 195-204.

Villalba, J.M. and P. Navas, Plasma membrane redox system in the control of stress-induced apoptosis. Antioxid Redox Signal, 2000. 2(2): p. 213-30.

Dixon, S.J., et al., Ferroptosis: an iron-dependent form of nonapoptotic cell death. Cell, 2012. 149(5): p. 1060-72.

Yang, W.S. and B.R. Stockwell, Ferroptosis: Death by Lipid Peroxidation. Trends Cell Biol, 2016. 26(3): p. 165-76.

Pasparakis, M. and P. Vandenabeele, Necroptosis and its role in inflammation. Nature, 2015. 517(7534): p. 311-20.

Yang, W.S., et al., Regulation of ferroptotic cancer cell death by GPX4. Cell, 2014. 156(1-2): p. 317-31.

Shimada, K., et al., Global survey of cell death mechanisms reveals metabolic regulation of ferroptosis. Nat Chem Biol, 2016. 12(7): p. 497-503.

Burdon, R.H., Superoxide and hydrogen peroxide in relation to mammalian cell proliferation. Free Radic Biol Med, 1995. 18(4): p. 775-94.

Pelicano, H., D. Carney, and P. Huang, ROS stress in cancer cells and therapeutic implications. Drug Resist Updat, 2004. 7(2): p. 97-110.

Szatrowski, T.P. and C.F. Nathan, Production of large amounts of hydrogen peroxide by human tumor cells. Cancer Res, 1991. 51(3): p. 794-8.

Tomasetti, M., et al., Redox-active and redox-silent compounds: synergistic therapeutics in cancer. Curr Med Chem, 2015. 22(5): p. 552-68.

Toyokuni, S., et al., Iron and thiol redox signaling in cancer: An exquisite balance to escape ferroptosis. Free Radic Biol Med, 2017. 108: p. 610-626.

Fang, J., H. Nakamura, and A.K. Iyer, Tumor-targeted induction of oxystress for cancer therapy. J Drug Target, 2007. 15(7-8): p. 475-86.

Trachootham, D., J. Alexandre, and P. Huang, Targeting cancer cells by ROS-mediated mechanisms: a radical therapeutic approach? Nat Rev Drug Discov, 2009. 8(7): p. 579-91.

Lockwood, K., S. Moesgaard, and K. Folkers, Partial and complete regression of breast cancer in patients in relation to dosage of coenzyme Q10. Biochem Biophys Res Commun, 1994. 199(3): p. 1504-8.

Chen, P.Y., et al., Protective effect of Co-enzyme Q10 On doxorubicin-induced cardiomyopathy of rat hearts. Environ Toxicol, 2017. 32(2): p. 679-689.

Papucci, L., et al., Coenzyme q10 prevents apoptosis by inhibiting mitochondrial depolarization independently of its free radical scavenging property. J Biol Chem, 2003. 278(30): p. 28220-8.

Brea-Calvo, G., et al., Chemotherapy induces an increase in coenzyme Q10 levels in cancer cell lines. Free Radic Biol Med, 2006. 40(8): p. 1293-302.

Harvie, M., Nutritional supplements and cancer: potential benefits and proven harms. Am Soc Clin Oncol Educ Book, 2014: p. e478-86.

Dixon, S.J., et al., Pharmacological inhibition of cystine-glutamate exchange induces endoplasmic reticulum stress and ferroptosis. Elife, 2014. 3: p. e02523.

Hayano, M., et al., Loss of cysteinyl-tRNA synthetase (CARS) induces the transsulfuration pathway and inhibits ferroptosis induced by cystine deprivation. Cell Death Differ, 2016. 23(2): p. 270-8.

Skouta, R., et al., Ferrostatins inhibit oxidative lipid damage and cell death in diverse disease models. J Am Chem Soc, 2014. 136(12): p. 4551-6.

Jiang, L., et al., Ferroptosis as a p53-mediated activity during tumour suppression. Nature, 2015. 520(7545): p. 57-62.

Ishimoto, T., et al., CD44 variant regulates redox status in cancer cells by stabilizing the xCT subunit of system xc(-) and thereby promotes tumor growth. Cancer Cell, 2011. 19(3): p. 387-400.

Ogunrinu, T.A. and H. Sontheimer, Hypoxia increases the dependence of glioma cells on glutathione. J Biol Chem, 2010. 285(48): p. 37716-24.

Guerriero, E., et al., GPX4 and GPX7 over-expression in human hepatocellular carcinoma tissues. Eur J Histochem, 2015. 59(4): p. 2540.

Wu, K.C., J.Y. Cui, and C.D. Klaassen, Beneficial role of Nrf2 in regulating NADPH generation and consumption. Toxicol Sci, 2011. 123(2): p. 590-600.

Sayin, V.I., et al., Antioxidants accelerate lung cancer progression in mice. Sci Transl Med, 2014. 6(221): p. 221ra15.

Freed-Pastor, W.A., et al., Mutant p53 disrupts mammary tissue architecture via the mevalonate pathway. Cell, 2012. 148(1-2): p. 244-58.

Gruenbacher, G. and M. Thurnher, Mevalonate metabolism in cancer. Cancer Lett, 2015. 356(2 Pt A): p. 192-6.

Jiang, P., et al., In vitro and in vivo anticancer effects of mevalonate pathway modulation on human cancer cells. Br J Cancer, 2014. 111(8): p. 1562-71.

Ortiz, T., et al., Amitriptyline down-regulates coenzyme Q10 biosynthesis in lung cancer cells. Eur J Pharmacol, 2017. 797: p. 75-82.

Acosta, M.J., et al., Coenzyme Q biosynthesis in health and disease. Biochim Biophys Acta, 2016. 1857(8): p. 1079-1085.

Stefely, J.A. and D.J. Pagliarini, Biochemistry of Mitochondrial Coenzyme Q Biosynthesis. Trends Biochem Sci, 2017. 42(10): p. 824-843.

Cerami, E., et al., The cBio cancer genomics portal: an open platform for exploring multidimensional cancer genomics data. Cancer Discov, 2012. 2(5): p. 401-4.

Gao, J., et al., Integrative analysis of complex cancer genomics and clinical profiles using the cBioPortal. Sci Signal, 2013. 6(269): p. pl1.

43. Poon, W.W., et al., Identification of Escherichia coli ubiB, a gene required for the first monooxygenase step in ubiquinone biosynthesis. J Bacteriol, 2000. 182(18): p. 5139-46.

Stefely, J.A., et al., Mitochondrial ADCK3 employs an atypical protein kinase-like fold to enable coenzyme Q biosynthesis. Mol Cell, 2015. 57(1): p. 83-94.

Hasegawa, M., et al., Functional interactions of the cystine/glutamate antiporter, CD44v and MUC1-C oncoprotein in triple-negative breast cancer cells. Oncotarget, 2016. 7(11): p. 11756-69.

Timmerman, L.A., et al., Glutamine sensitivity analysis identifies the xCT antiporter as a common triple-negative breast tumor therapeutic target. Cancer Cell, 2013. 24(4): p. 450-65.

Viswanathan, V.S., et al., Dependency of a therapy-resistant state of cancer cells on a lipid peroxidase pathway. Nature, 2017. 547(7664): p. 453-457.

Horvath, R., et al., Adult-onset cerebellar ataxia due to mutations in CABC1/ADCK3. J Neurol Neurosurg Psychiatry, 2012. 83(2): p. 174-8.

Mollet, J., et al., CABC1 gene mutations cause ubiquinone deficiency with cerebellar ataxia and seizures. Am J Hum Genet, 2008. 82(3): p. 623-30.

Stefely, J.A., et al., Cerebellar Ataxia and Coenzyme Q Deficiency through Loss of Unorthodox Kinase Activity. Mol Cell, 2016. 63(4): p. 608-20.

Stancu, C. and A. Sima, Statins: mechanism of action and effects. J Cell Mol Med, 2001. 5(4): p. 378-87.

Quinzii, C.M., et al., Effects of inhibiting CoQ10 biosynthesis with 4-nitrobenzoate in human fibroblasts. PLoS One, 2012. 7(2): p. e30606.

Yang, W.S., et al., Peroxidation of polyunsaturated fatty acids by lipoxygenases drives ferroptosis. Proc Natl Acad Sci USA, 2016. 113(34): p. E4966-75.

Junker, B.H., Flux analysis in plant metabolic networks: increasing throughput and coverage. Curr Opin Biotechnol, 2014. 26: p. 183-8.

Asquith, C.R.M., et al., SGC-GAK-1: A Chemical Probe for Cyclin G Associated Kinase (GAK). J Med Chem, 2019. 62(5): p. 2830-2836.

Dent, R., et al., Triple-negative breast cancer: clinical features and patterns of recurrence. Clin Cancer Res, 2007. 13(15 Pt 1): p. 4429-34.

Welsch, M.E., et al., Multivalent Small-Molecule Pan-RAS Inhibitors. Cell, 2017. 168(5): p. 878-889 e29.

Kaplan, A., et al., Small molecule-induced oxidation of protein disulfide isomerase is neuroprotective. Proc Natl Acad Sci U S A, 2015. 112(17): p. E2245-52.

Wheeler, B. and Z. Jia, Preparation and characterization of human ADCK3, a putative atypical kinase. Protein Expr Purif, 2015. 108: p. 13-17.

Jafari, R., et al., The cellular thermal shift assay for evaluating drug target interactions in cells. Nat Protoc, 2014. 9(9): p. 2100-22.

Martinez Molina, D. and P. Nordlund, The Cellular Thermal Shift Assay: A Novel Biophysical Assay for In Situ Drug Target Engagement and Mechanistic Biomarker Studies. Annu Rev Pharmacol Toxicol, 2016. 56: p. 141-61.

Pai, M.Y., et al., Drug affinity responsive target stability (DARTS) for small-molecule target identification. Methods Mol Biol, 2015. 1263: p. 287-98.

Krainz, T., et al., A Mitochondrial-Targeted Nitroxide Is a Potent Inhibitor of Ferroptosis. ACS Cent Sci, 2016. 2(9): p. 653-659.

Di, L., et al., High throughput artificial membrane permeability assay for blood-brain barrier. Eur J Med Chem, 2003. 38(3): p. 223-32.

Kansy, M., F. Senner, and K. Gubernator, Physicochemical high throughput screening: parallel artificial membrane permeation assay in the description of passive absorption processes. J Med Chem, 1998. 41(7): p. 1007-10.

Hanahan, D. and R.A. Weinberg, Hallmarks of cancer: the next generation. Cell, 2011. 144(5): p. 646-74.

Takats, Z., et al., Mass spectrometry sampling under ambient conditions with desorption electrospray ionization. Science, 2004. 306(5695): p. 471-3.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A method for treating or ameliorating the effects of a disorder in a subject, comprising administering to the subject an effective amount of an agent that increases lipid-based reactive oxygen species (ROS).

2. The method of claim 1, wherein the disorder is associated with accumulation of an oncolipid.

3. The method of claim 2, wherein the oncolipid is an antioxidant.

4. The method of claim 3, wherein the antioxidant is endogenous to the subject.

5. The method of claim 3, wherein the antioxidant is coenzyme Q₁₀ (CoQ₁₀).

6. The method of claim 1, wherein the disorder is a cancer.

7. The method of claim 6, wherein the cancer is selected from the group consisting of head and neck cancer, prostate cancer, stomach cancer, colorectal cancer, bladder cancer, thymoma, thymic carcinoma, lung adenocarcinoma, uterine carcinosarcoma, cervical carcinosarcoma, esophageal carcinosarcoma, non-small-cell lung carcinoma (NSCLC), pancreatic cancer, breast cancer, melanoma, diffuse large B-cell lymphoma (DLBCL), ovarian cancer, liver cancer, chronic lymphocytic leukemia (CLL), cholangiocarcinoma, neuroendocrine prostate cancer (NEPC), and combinations thereof.

8. The method of claim 1, wherein the disorder is breast cancer.

9. The method of claim 8, wherein the breast cancer is mesenchymal breast cancer.

10. The method of claim 8, wherein the breast cancer is triple-negative breast cancer.

11. The method of claim 8, wherein the breast cancer is unresectable.

12. The method of claim 1, wherein the subject is a mammal.

13. The method of claim 12, wherein the mammal is selected from the group consisting of humans, veterinary animals, and agricultural animals.

14. The method of claim 1, wherein the subject is a human.

15. The method of claim 1, wherein the disorder is associated with overexpression of ADCK3.

16. The method of claim 1, wherein the agent increases lipid-based reactive oxygen species (ROS) by inhibiting coenzyme Q₁₀ (CoQ₁₀) production.

17. The method of claim 1, wherein the agent is an ADCK3 inhibitor.

18. The methd of claim 17, wherein the ADCK3 inhibitor is selected from dasatinib, PD-173955, R406, TG-100-115, UNC-CA157, SGC-GAK-1, pharmaceutical compositions thereof and combinations thereof.

19. The method of claim 17, wherein the ADCK3 inhibitor is SGC-GAK-1 or a pharmaceutical composition thereof.

20. The method of claim 1, further comprising co-administering to the subject an effective amount of a ferroptosis inducer selected from the group consisting of erastin, imidazole ketone erastin (IKE), piperazine erastin (PE), sulfasalazine, sorafenib, RSL3, ferroptosis inducer 56 (FIN56), caspase-independent lethal 56 (CIL56), ferroptosis inducer endoperoxide (FINO₂), pharmaceutical compositions thereof and combinations thereof.

21. The method of claim 1, further comprising co-administering to the subject a therapy selected from the group consisting of surgery, chemotherapy, radiation therapy, immunotherapy, and combinations thereof.

22. The method of claim 21, wherein the chemotherapy comprises administering to the subject a therapeutically useful chemotherapeutic agent.

23. The method of claim 22, wherein the chemotherapeutic agent is selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, pharmaceutical compositions thereof and combinations thereof.

24. The method of claim 21, wherein the immunotherapy comprises administering to the subject a therapeutically useful immunotherapeutic agent.

25. The method of 24, wherein the immunotherapeutic agent comprises chimeric antigen receptor (CAR) T-cell therapeutics, T-cell receptor (TCR) therapeutics, tumor-infiltrating lymphocyte (TIL) therapeutics, monoclonal antibody therapeutics, immune checkpoint inhibitors and combinations thereof.

26. The method of claim 24, wherein the immunotherapeutic agent is selected from the group consisting of ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab, pharmaceutical compositions thereof and combinations thereof.

27. A method for treating a subject with a cancer that is sensitive to an oncolipid-targeting therapy, comprising the steps of:
(a) determining the expression levels of ADCK3 and ADCK4 in a biological sample from the subject;
(b) identifying the subject as having a cancer that is sensitive to an oncolipid-targeting therapy, if the level of ADCK3 determined in step (a) is significantly higher than a first predetermined reference, and the level of ADCK4 determined in step (a) is significantly lower than a second predetermined reference; and
(c) treating the subject identified in step (b) as having a cancer sensitive to an oncolipid-targeting therapy with the oncolipid-targeting therapy.

28. The method of claim 27, wherein the first predetermined reference is the expression level of ADCK3 in normal tissue of the subject, and the second predetermined reference is the expression level of ADCK4 in normal tissue of the subject.

29. The method of claim 27, wherein the oncolipid is coenzyme Q₁₀ (CoQ₁₀).

30. The method of claim 27, wherein the oncolipid-targeting therapy comprises administering to the subject an effective amount of an ADCK3 inhibitor.

31. The method of claim 30, wherein the ADCK3 inhibitor is selected from dasatinib, PD-173955, R406, TG-100-115, UNC-CA157, SGC-GAK-1, pharmaceutical compositions thereof and combinations thereof.

32. The method of claim 30, wherein the ADCK3 inhibitor is SGC-GAK-1 or a pharmaceutical composition thereof.

33. The method of claim 27, wherein the cancer is selected from the group consisting of head and neck cancer, prostate cancer, stomach cancer, colorectal cancer, bladder cancer, thymoma, thymic carcinoma, lung adenocarcinoma, uterine carcinosarcoma, cervical carcinosarcoma, esophageal carcinosarcoma, non-small-cell lung carcinoma (NSCLC), pancreatic cancer, breast cancer, melanoma, diffuse large B-cell lymphoma (DLBCL), ovarian cancer, liver cancer, chronic lymphocytic leukemia (CLL), cholangiocarcinoma, neuroendocrine prostate cancer (NEPC), and combinations thereof.

34. The method of claim 27, wherein the cancer is breast cancer.

35. The method of claim 34, wherein the breast cancer is mesenchymal breast cancer.

36. The method of claim 34, wherein the breast cancer is triple-negative breast cancer.

37. The method of claim 34, wherein the breast cancer is unresectable.

38. The method of claim 27, wherein the subject is a mammal.

39. The method of claim 38, wherein the mammal is selected from the group consisting of humans, veterinary animals, and agricultural animals.

40. The method of claim 27, wherein the subject is a human.

41. A method for modulating coenzyme Q₁₀ (CoQ₁₀) level in a subject, comprising:
(a) determining a baseline CoQ₁₀ level in the subject;
(b) administering to the subject an effective amount of an ADCK3 inhibitor; and
(c) determining whether the baseline CoQ₁₀ level in the subject has changed.

42. The method of claim 41, wherein the ADCK3 inhibitor is SGC-GAK-1 or a pharmaceutical composition thereof.

43. The method of claim 41, wherein the CoQ₁₀ levels in the subject are determined by LC-MS.

44. The method of claim 41, wherein the measured CoQ₁₀ levels include reduced, oxidized, and/or total cellular CoQ₁₀ levels.
